(19) United States
(12) Patent Application Publication
(10) Pub. No.: US 2015/0122056 A1
(43) Pub. Date: May 7, 2015

(54) cGMP COMPLIANT INDELIBLE INK PENS, AND METHODS OF USING THE SAME

(71) Applicant: Kymanox, LLC, Durham, NC (US)

(72) Inventors: Stephen M. PERRY, Cary, NC (US); Milan DRAGIC, Cary, NC (US)

(21) Appl. No.: 14/590,992

(22) Filed: Jan. 7, 2015

Related U.S. Application Data
(63) Continuation of application No. 12/701,561, filed on Feb. 6, 2010, now abandoned.
(60) Provisional application No. 61/150,736, filed on Feb. 7, 2009.

(51) Int. Cl. B43K 29/007; G01N 33/32

(52) U.S. Cl. CPC B43K 29/007; G01N 33/32
USPC 73/865.8

(57) ABSTRACT

The present invention conveniently and practically addresses a significant need. The invention provides pens that are clearly labeled with a phrase to indicate that the pens contain GMP-compliant indelible ink. In one aspect, the invention provides an apparatus for writing, comprising: (i) indelible ink; (ii) a construction suitable for containing the indelible ink; and (iii) a text-based label on an outer surface of the construction, wherein the label comprises a word or phrase intended to validate the apparatus for GMP compliance, FDA compliance, and/or EMEA compliance. In another aspect, a method is disclosed for providing a writing procedure that is GMP-compliant, FDA-compliant, and/or EMEA-compliant. In yet another aspect, the invention describes a method for labeling a pen containing indelible ink.

### PRIORITY DATA

This patent application is a continuation application of U.S. patent application Ser. No. 12/701,561, filed Feb. 6, 2010, which is a non-provisional application of U.S. Provisional Patent Application No. 61/150,736, filed Feb. 7, 2009, each of which is hereby incorporated herein by reference for all purposes.

### FIELD OF THE INVENTION

The present invention generally relates to methods and apparatus for compliance with current Good Manufacturing Practice (cGMP) in the biotechnology, pharmaceutical, medical, medical device, and food industries.

### BACKGROUND OF THE INVENTION

“Good Manufacturing Practice” or “GMP” (also referred to as “current Good Manufacturing Practice” or “cGMP”) is a term that is recognized worldwide for the control and management of manufacturing and quality-control testing of pharmaceutical, medical, biotechnology, and food products. As used herein, cGMP and GMP are equivalent terms. In the United States, GMP is contained in §501(B) of the 1938 Food, Drug, and Cosmetic Act (21 USC 351). Current GMP regulations are also set forth in the U.S. Code of Federal Regulations (CFR) at 21 CFR Parts 110, 210-211, and 820.

GMP takes the holistic approach of regulating the manufacturing and laboratory-testing environment itself. An important part of GMP is documentation of every aspect of the process, activities, and operations involved with manufacture. If the documentation showing how a product was made and tested (which enables traceability) is not correct and in order, then the product does not meet the required specification and is considered contaminated and/or adulterated. Additionally, GMP requires that all manufacturing and testing equipment has been qualified as suitable for use, and that all operational methodologies and procedures (such as manufacturing, cleaning, and analytical testing) utilized in the process have been validated (according to predetermined specifications), to demonstrate that they can perform their purported function(s).

GMPs are enforced in the United States by the Food and Drug Administration (FDA), an agency of the United States Department of Health and Human Services. Within the European Union, GMP inspections are performed by National Regulatory Agencies. The European Medicines Agency, also known as the European Medicines Evaluation Agency (EMEA), is a European agency for the evaluation of medicinal products. The EMEA is, generally speaking, parallel to the FDA, but without FDA-style centralization. GMP-type inspections are performed in the United Kingdom by the Medicines and Healthcare products Regulatory Agency (MHRA); in Australia by the Therapeutical Goods Administration (TGA); in India by the Ministry of Health; and by similar national organizations worldwide. The International Conference on Harmonization (ICH) brings together the regulatory authorities of Europe, Japan, and the United States and experts from the pharmaceutical industry to discuss scientific and technical aspects of pharmaceutical product registration.

The FDA, EMEA, ICH, and similar government bodies worldwide require that a document be recorded with indelible ink. When non-indelible ink (e.g., gel ink) is used, the ink can become erased or otherwise damaged, thereby jeopardizing the information on the written document. These non-GMP-compliant recording procedures are known to cause significant economic damages to the parties involved.

Companies can benefit from a way to easily identify and use pens that are GMP-compliant. Time and money would be saved, by reducing the amount of exception reports caused by improper documentation techniques. What is therefore needed in the industry is a method of supplying and appropriately labeling GMP-compliant pens that contain proper indelible ink for GMP procedures. A solution to this significant problem should be practical and able to be readily implemented across the biotechnology, pharmaceutical, medical, medical device, and food industries.

### SUMMARY OF THE INVENTION

The present invention, as described herein, conveniently and practically addresses these known needs in the art. The methods of the invention provide pens that are clearly labeled with certain words (as described below) indicating the pen contains indelible ink and is GMP-compliant.

In some variations, this invention provides an apparatus for writing, the apparatus comprising:

(i) indelible ink;

(ii) a construction (e.g., a pen) suitable for containing the indelible ink and for applying the indelible ink to a writing surface; and

(iii) a text-based label on an outer surface of the construction, wherein the label comprises a phrase to validate the apparatus for GMP compliance, FDA compliance, or EMEA compliance.

The indelible ink can be blue or black. In some embodiments, the text-based label comprises the acronym “GMP” or “cGMP” within the phrase. For example, the phrase can be “GMP Compliant Indelible Ink” or “cGMP Compliant Indelible Ink” applied on the outer surface. In some embodiments, the text-based label comprises the acronym “FDA” within the phrase.

Variations of the invention provide a method for providing a writing procedure that is GMP-compliant, FDA-compliant, or EMEA-compliant, the method comprising providing a pen containing indelible ink (e.g., blue or black indelible ink), wherein an outer surface of the pen includes a text-based label comprising a phrase to validate the pen for compliance with GMP, FDA, or EMEA.

In some embodiments of this method, the text-based label comprises the acronym “GMP” or “cGMP” within the phrase. For example, the phrase can be “GMP Compliant Indelible Ink” or “cGMP Compliant Indelible Ink” on the outer surface. The text-based label can include the acronym “FDA” within the phrase.

Other variations of the invention provide a method for labeling a pen containing indelible ink, the method comprising:

(i) providing a pen containing indelible ink;

(ii) selecting a phrase that indicates the pen is GMP-compliant, FDA-compliant, or EMEA-compliant; and

(iii) attaching a label comprising the selected phrase from step (ii) onto the pen from step (i).

In some embodiments, the label includes a color that substantially matches the color of the indelible ink.

The label can be attached by a means selected from the group consisting of screen printing, etching, pressing, and embedding. The label can be printed on an attachable sleeve that comprises the label. This attachable sleeve is optionally applied to a pen previously used for GMP, FDA, or EMEA compliance.

The method can further include marketing or selling one or more GMP-compliant pens as provided herein.

### BRIEF DESCRIPTION OF THE DRAWINGS

FIG. 1 is an illustrative depiction of one embodiment of the invention (Standard Clic-Stic, Blue or Black), wherein the phrase “cGMP Compliant Indelible Ink” is used to indicate that the ink pen is GMP-compliant.

FIG. 2 is an illustrative depiction of one embodiment of the invention (Widebody, Blue or Black), wherein the phrase “cGMP Compliant Indelible Ink” is used to indicate that the ink pen is GMP-compliant.

FIG. 3 is an illustrative depiction of one embodiment of the invention (Triangle Barrel, Blue or Black), wherein the phrase “cGMP Compliant Indelible Ink” is used to indicate that the ink pen is GMP-compliant.

FIG. 4 is an illustrative depiction of one embodiment of the invention (Comfort Grip, Blue or Black), wherein the phrase “cGMP Compliant Indelible Ink” is used to indicate that the ink pen is GMP-compliant.

### DETAILED DESCRIPTION OF EMBODIMENTS OF THE INVENTION

The apparatus and methods of the present invention will now be described in detail by reference to various non-limiting embodiments of the invention.

Unless otherwise indicated, all numbers expressing dimensions, parameters, and so forth used in the specification and claims are to be understood as being modified in all instances by the term “about.” Without limiting the application of the doctrine of equivalents to the scope of the claims, each numerical parameter should at least be construed in light of the number of significant digits and by applying ordinary rounding techniques.

As used in this specification and the appended claims, the singular forms “a,” “an,” and “the” include plural referents unless the context clearly indicates otherwise.

Certain preferred embodiments of the present invention will be described in more detail, including reference to the accompanying figures. The figures are understood to provide representative illustration of the invention and are not limiting in their content or scale. It will be understood by one of ordinary skill in the art that the scope of the invention extends beyond the specific embodiments depicted. This invention also incorporates routine experimentation and optimization of the methods and apparatus described herein.

“Good Manufacturing Practice” (GMP) or “Current Good Manufacturing Practice” (cGMP) are widely recognized terms for the control and management of manufacturing and quality-control testing of pharmaceutical, medical, biotechnology, and food products. As used herein, cGMP and GMP are equivalent terms. In the United States, GMP is contained in §501(B) of the 1938 Food, Drug, and Cosmetic Act (21 USC 351), which is hereby incorporated by reference herein in its entirety.

A record is a document that recalls or relates past events. It is a body of known facts. The records a company keeps to comply with GMP regulations concern the people, the equipment, and the step-by-step processes of manufacturing a quality product. GMP regulations require that a company keep complete and accurate records about its products. Because GMP regulations have the force of law, the FDA (or similar governmental body outside the U.S.) has a right to examine these records when they inspect a plant. Poor and inadequate records are violations of the law and are often cited by FDA inspectors in reports of plant inspections. As will be appreciated, it is also good business practice to keep accurate records, for a variety of reasons such as avoiding errors that can lead to costly recalls or customer complaints.

GMP regulations require that records be kept for each step of a manufacturing process, from the time components enter the plant as raw materials until the finished product is distributed. The regulation also requires recording the results of all laboratory tests necessary to assure that one complies with established specifications and standards. Some required records include: product master records; batch records; material/component control records; equipment logs; and personnel records.

Importantly, GMP regulations require that such recording be accomplished using indelible ink. As is generally known, an “ink” is a liquid containing various pigments and/or dyes used for coloring a surface to produce an image or text. Ink is a complex medium, comprising (in various concentrations) solvents, pigments, dyes, resins, lubricants, solubilizers, particulate matter, fluorescers, and/or other materials. The components of inks serve many purposes. The carrier, colorants, and other additives are typically used to control flow, thickness, and appearance of the ink when dry.

An “indelible ink” is an ink that cannot be easily removed, i.e. it cannot be erased or washed away under normal conditions. Indelible ink can contain dyes, pigments, surface active agents (e.g., a dispersant compatible with the pigment), organic solvents, and other chemicals. An indelible ink includes an amount of an ink capable of producing a visible writing on a substrate such as natural or synthetic paper. Typically, an indelible ink includes about 5 wt % to 50 wt % of one or more dyes or pigments. Higher or lower amount of dyes and pigments may be used, depending upon the specific dye's molecular weight and its propensity to produce a visible image on the substrate, or for cost reasons. An indelible ink can have various colors but typically includes at least one of blue, black, violet, or red dye. The indelible ink should be capable of being photocopied, pursuant to cGMP and related regulations. Blue and black indelible inks are preferred, although not required, in the context of the present invention.

For the purposes of the present invention, “indelible” and “substantially indelible” are taken as the same. It will be appreciated by a skilled artisan that even indelible inks can generally be removed with appropriate solvents under suitable conditions (such as extreme temperatures and pH) or by physical removal of the ink. For the present invention, it is intended that the indelible ink would not run if a common solvent (such as water or an aqueous alcohol solution) spilled onto a piece of paper comprising dried indelible ink. A “run” of ink generally means that a significant amount of ink dispersion has occurred, possibly causing loss in previously recorded information. At the microscopic level, some amount of molecular dispersion can occur in the presence of water or other solvents, but this amount of dispersion may not be readily apparent to a person viewing the document.

An exemplary indelible ink is India Ink. Other examples of indelible inks include those described in U.S. Pat. No. 5,449,400 or U.S. Pat. No. 6,258,873, which are both hereby incorporated by reference herein. The invention described herein is by no means limited to any particular selection or source of indelible ink. Notwithstanding the foregoing, some indelible inks are preferred due to certain chemicals and solvents that may be more prevalent in GMP manufacturing facilities.

Variations of the invention provide indelible-ink pens that are clearly labeled as GMP-compliant. As used herein, a “pen” is any apparatus of suitable construction that is capable of containing indelible ink and is further capable of being labeled with a phrase as provided herein. For convenience, the term “pen” is used herein to refer to any suitable construction as a writing instrument that can apply ink to a surface, usually (but not necessarily) paper.

A pen provided by the present invention is clearly labeled with a textual phrase that is intended to indicate that the pen is GMP-compliant and that it contains indelible ink. In some preferred embodiments, the phrase is “cGMP Compliant Indelible Ink.” In some embodiments, the phrase is “GMP Compliant Indelible Ink Pen.” In other embodiments, the phrase is “GMP Pen Indelible Ink.” Again noting that cGMP and GMP are equivalent terms, any instance of “GMP” can be replaced by “cGMP” (and vice-versa) without loss of generality and with no difference in meaning or intent.

In some embodiments, a selected phrase is, or comprises, a trademark (whether registered or unregistered). Therefore, the symbol ™ or the symbol ® may optionally appear along with a selected phrase.

A selected phrase can be repeated multiple times on a single pen, so that the pen is more readily identified as GMP-compliant. In various embodiments, a phrase can be repeated two, three, four, or more times. Multiple lines of text can be placed adjacently, in the axial dimension; adjacently, in the radial dimension; or can be placed in any other configuration on the pen. Optionally, more than one distinct phrase can be utilized on a single pen. Of course, it is also possible to use one or more phrases as provided herein, coupled with some other word, phrase, logo, or the like, such as a company name or logo.

Preferred embodiments employ black indelible ink or blue indelible ink, pursuant to GMP and related regulations. In certain embodiments, a pen is designed with dual ink containers and a mechanism to switch between blue and black ink, depending on user preference or various guidelines. Optionally, additional inks can be included in a multiple-ink pen. For example, a chamber with red indelible ink could be included, intended for instance for markups on documents.

Pens can be constructed with a variety of sizes, colors, and styles as desired. Pens can be retractable (using a click button or by other means). Pens can be provided with or without a cap or other means of isolating the ink tip when not in use.

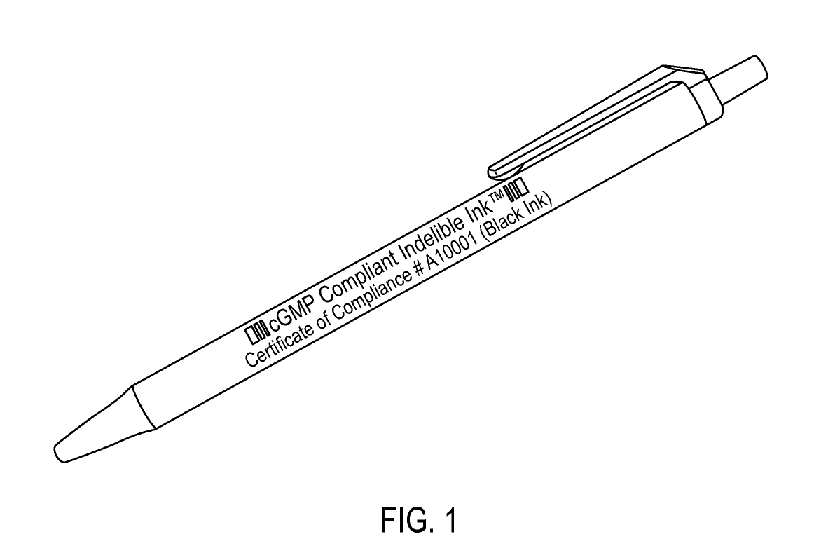

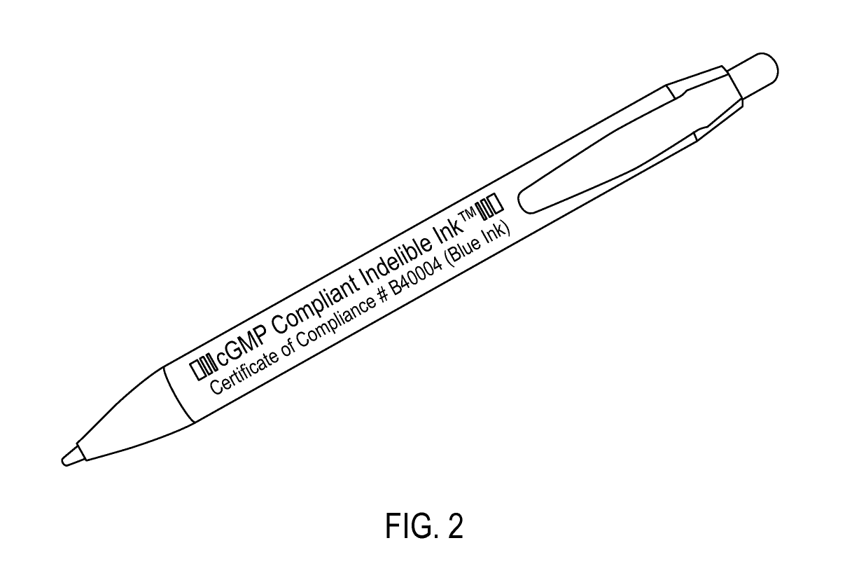

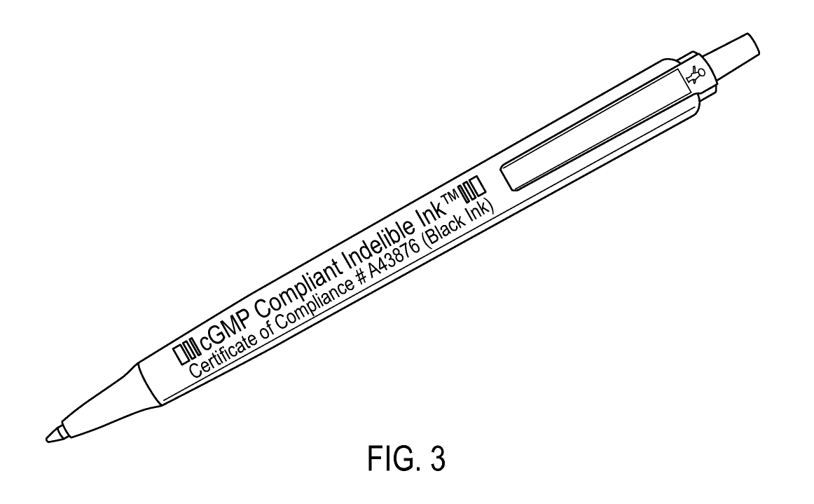

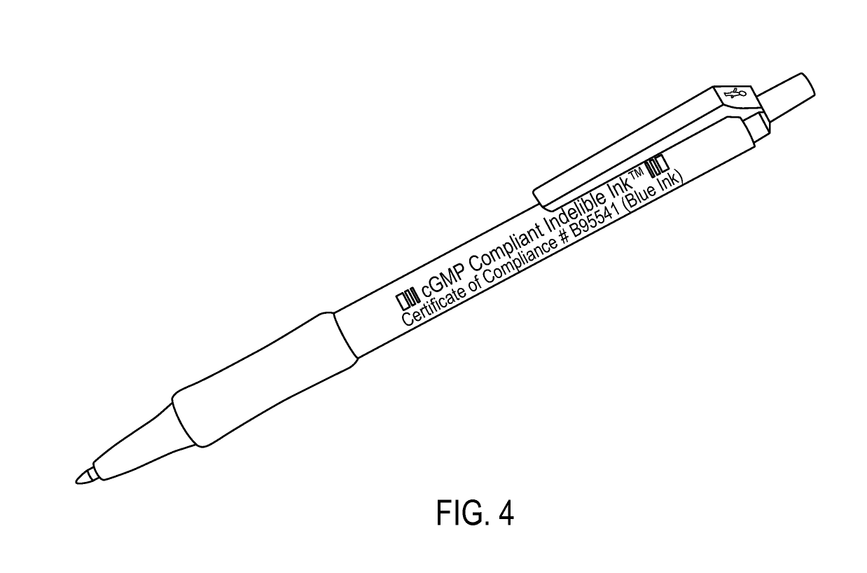

Pens can be, for example, of standard size (FIG. 1) or widebody (FIG. 2). Pens can utilize a triangle barrel (FIG. 3) in some embodiments. A comfort grip (as in FIG. 4) can be included if desired. FIGS. 1-4 depict certain exemplary variations of the invention. These figures are by no means limiting of the present invention.

The external appearance of a pen can be designed to coordinate with the ink color, if desired. For example, pens with blue indelible ink can employ an exterior comprising a blue color, either as accents or substantially completely blue.

The label (phrase) itself can be blue, or the label can be white on a blue background, and so on. Generally speaking, the phrases indicating GMP compliance can be printed in black and white or in any color combinations. A variety of design styles (text font, size, etc.) can be used for the label text.

A phrase can be attached to a pen in several different ways, such as screen printing, etching, pressing, and embedding, for example. In some embodiments, a label can be attached to a pen by an attachable sleeve that comprises the label. The sleeve can be attached to a pen by use of an adhesive or by other known means.

Pens can be sold (or otherwise provided) individually or, more typically, packaged and sold as packs of 10, 20, 50, 100 or more pens. Pens can be sold or provided by any known means, including in physical stores or by e-commerce via the Internet (e.g., GMPPENS.com). It is preferable to issue a certificate of compliance, or similar document, along with a sale of one or more pens. The certificate of compliance should include an identification number for documentation and audit purposes. Optionally, this identification number is printed on the GMP-compliant pens.

In some methods of the invention, each lot of pens is tested for quality and compliance. Then a certificate of compliance is generated to document acceptance of the lot before it is shipped.

According to the invention, pens can be labeled with a variety of phrases. In various embodiments, the phrase comprises one or more of the following words or text strings:

GMP Compliant Ink Pen

GMP Compliant

GMP Comply

GMP Confirmed

GMP Accommodating

GMP Standard

GMP Accepted

GMP Approved

GMP Regulation

GMP Model

GMP Prototype

GMP Validated

GMP Authenticated

GMP Certified

GMP Documented

GMP Verified

GMP Ready

GMP Accessible

GMP Arranged

GMP Available

GMP Qualified

GMP Act

GMP Operate

GMP Officiate

GMP Function

GMP International

GMP Global

GMP Universal

GMP Impact

GMP Influence

GMP Current

GMP Common

GMP Knowledge

GMP Established

GMP Conventional

GMP Comply

GMPly

GMP Confirmed

GMP Firm

GMPaffirmed

GaccoMmodatingP

StandardGMP

GMaPproved

GMPreg

GMPro

GMPrototype

GMPalidated

GMPhenticated

GMPfied

GMPdocumented

VeriGMP

GMPready

ReadyGMP

GMPAct

ActGMP

OperateGMP

FuctionGMP

GlobalMP

GlobalGMP

GMPuniversal

GiMPact

GMPfluence

GMP Technology

GMP Tech

Technology GMP

In other embodiments of the invention, the phrase comprises one or more of the following words or text strings:

FDA&EMEA Compliant

FDA&EMEA Comply

FDA&EMEA Confirmed

FDA&EMEA Affirm

FDA&EMEA Approved

FDAdaptable

FDAdapted

EMEAdaptable

EMEA Adapted

AffDA

FDA Pros

EMEA Pros

FDApproved

EMEApproved

In this detailed description, reference has been made to multiple embodiments and to the accompanying drawings in which is shown by way of illustration specific exemplary embodiments in which the invention may be practiced. These embodiments are described in sufficient detail to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

All publications, patents, and patent applications cited in this specification are herein incorporated by reference in their entirety as if each publication, patent, or patent application were specifically and individually put forth herein.

The embodiments, variations, and figures described above should provide an indication of the utility and versatility of the present invention. Of course, many more devices can be developed that involve combinations of device elements as provided herein. Other embodiments that do not provide all of the features and advantages set forth herein may also be utilized, without departing from the spirit and scope of the present invention. Such modifications and variations are considered to be within the scope of the invention defined by the appended claims.

What is claimed is:

1. A method for testing and labeling a pen containing indelible ink for documentation and audit purposes, said method comprising:
(i) constructing a pen containing indelible ink;
(ii) testing said pen for quality and compliance with GMP, FDA, or EMEA regulations;
(iii) generating a certificate of compliance to document acceptance of said pen, wherein said certificate of compliance includes an identification number;
(iv) selecting a phrase that indicates said pen is GMP-compliant, FDA-compliant, or EMEA-compliant; and
(v) attaching a label comprising said selected phrase from step (iv) onto said pen from step (i), wherein said label further comprises said identification number from step (iii).

2. The method of claim 1, wherein said phrase comprises the acronym “GMP” or “cGMP”.

3. The method of claim 2, wherein said phrase is “GMP Compliant Indelible Ink” or “cGMP Compliant Indelible Ink”.

4. The method of claim 1, wherein said phrase comprises the acronym “FDA”.

5. The method of claim 1, wherein said label includes a color that substantially matches the color of said indelible ink.

6. The method of claim 1, wherein said label is attached by a means selected from the group consisting of screen printing, etching, pressing, and embedding.

7. The method of claim 1, wherein said label is printed on an attachable sleeve that comprises said label.

8. The method of claim 7, wherein said attachable sleeve is applied to a pen previously used for GMP, FDA, or EMEA compliance.

9. The method of claim 1, said method further comprising marketing or selling said pen.
